(19) United States
(12) Patent Application Publication
(10) Pub. No.: US 2013/0116174 A1
(43) Pub. Date: May 9, 2013

(54) COMPOSITIONS AND METHODS FOR INCREASING POULTRY HATCHABILITY AND EARLY PERFORMANCE

(71) Applicants: Billy Hargis, Fayetteville, AR (US); Walter Bottje, Fayetteville, AR (US)

(72) Inventors: Billy Hargis, Fayetteville, AR (US); Walter Bottje, Fayetteville, AR (US)

(73) Assignee: The Board of Trustees of the University of Arkansas, Little Rock, AR (US)

(21) Appl. No.: 13/809,450

(22) PCT Filed: Jul. 11, 2011
(86) PCT No.: PCT/US11/43558

§ 371 Date: Jan. 10, 2013

Related U.S. Application Data
(60) Provisional application No. 61/363,246, filed on Jul. 11, 2010.

(51) Int. Cl. A61K 38/17; A61K 31/718

(52) U.S. Cl. CPC A61K 38/1703; A61K 31/718
USPC 514/5.7

(57) ABSTRACT

Compositions and methods of improving poultry hatch health are provided herein. Poultry hatch health and hatchability may be improved by administering compositions in ovo prior to hatch. The compositions include iodinated tyrosine-containing proteins, suitably iodinated casein, and carbohydrates.

### CROSS-REFERENCE TO RELATED APPLICATIONS

This patent application claims the benefit of priority of U.S. Provisional Patent Application No. 61/363,246, filed Jul. 11, 2010, which is incorporated herein by reference in its entirety.

### BACKGROUND

In commercial hatcheries, poults will hatch over a 48 h period of time and often may be transported considerable distance before having access to feed and water. Without access to feed and water, there can be long lasting deleterious effects on health and growth performance that include retardation of immune system development, ketoacidosis and extremely poor poult quality such that these severely metabolically challenged poults exhibit a generalized failure to thrive and grow and many die. Batal and Parsons (2002, Poult. Sci. 81:853-859) and Henderson et al. (2008, Inter. J. Poult. Sci. 7:211-214) have demonstrated that early nutritional supplementation improved growth performance in broilers. Halvey et al. (2003, J. Nutr. 133:1376-1382) reported that turkey poults exhibited increased amounts of satellite cells and skeletal muscle growth in response to early feeding immediately after hatch leading to enhanced myoblast development in embryos. Thus, early nutrition has been shown to be beneficial for early hatchling performance in broilers and turkeys.

### SUMMARY OF THE INVENTION

Compositions and methods of improving or increasing the health and hatchability of poultry are provided herein. In one aspect, compositions including an iodinated tyrosine-containing protein and a carbohydrate are provided.

In another aspect, methods of increasing the health of poultry hatchlings by administering compositions including an iodinated tyrosine-containing protein to poultry embryos in ovo are provided. Administration may increase the hatchability of the egg, may increase hatchling weight at hatch, may decrease mortality post-hatch and may increase weight, gain after hatch.

### BRIEF DESCRIPTION OF THE FIGURES

FIG. 1 is a graph showing the body weight (BW) in grams of chicken hatchlings on the date of hatch after administration of the indicated compositions at 2 days prior to hatch. The results are broken into the top 25%, middle 50% and bottom 25% by hatch weight.

FIG. 2 is a graph showing the body weight (BW) in grams of chicken hatchlings seven days after hatch after administration of the indicated compositions at 2 days prior to hatch. The results are broken into the top 25%, middle 50% and bottom 25% by hatch, weight.

### DETAILED DESCRIPTION

Compositions and methods for increasing the health of poultry hatchlings are provided herein. The compositions include an iodinated tyrosine-containing protein and suitably a carbohydrate. The iodinated tyrosine-containing protein may be any protein capable of being iodinated on tyrosines. Suitably the tyrosine-containing protein has at least two iodine molecules per mole of tyrosine, more suitably at least three, four, five or more iodine molecules per mole of tyrosine are present in the protein. In one embodiment, all of the tyrosines in the protein are iodinated. In another embodiment, iodination is not complete and only some tyrosines are iodinated.

Suitably, the tyrosine-containing protein is casein. Casein is a 212 amino acid protein that may be purified from milk and is rich in tyrosine. Casein and other tyrosine-containing proteins can be iodinated by methods known to those of skill in the art, including the methods used to make the iodinated casein described in the Examples. The compositions may contain at least two, suitably three or more iodines per protein molecule. Those of skill in the art will appreciate other proteins may be used in place of casein. Without being limited by theory, the iodinated tyrosine containing protein may function to improve poultry hatch health by stimulating metabolism through a thyroid-like effect of the iodinated protein as it is digested.

The composition may have between 5 μg/mL and 1 mg/mL of the iodinated tyrosine-containing protein. Suitably the composition has at least 10 μg/mL, 20 μg/mL, 30 μg/mL, 40 μg/mL, 50 μg/mL, 60 μg/mL 75 μg/mL, 85 μg/mL, 100 μg/mL, 150 μg/mL, 200 μg/mL, 250 μg/mL, 300 μg/mL, 350 μg/mL, or 375 μg/mL of the iodinated tyrosine-containing protein. Suitably the composition has less than 900 μg/mL, 800 μg/mL, 750 μg/mL, 700 μg/mL, 650 μg/mL, 600 μg/mL, 550 μg/mL, 500 μg/mL, 450 μg/mL, or 400 μg/mL of the iodinated tyrosine-containing protein. The amounts of protein provided may be combined to provide suitable ranges of the protein in the compositions. The iodination efficiency or the number of tyrosines in the protein may change the potency of the composition and thus affect the amount of protein in the composition required to deliver an effective dose.

The carbohydrate used in the composition is suitably soluble at 4° C. and injectable. In this case, the term ‘injectable’ relates only to the ability of the solution of carbohydrate and iodinated tyrosine-containing protein to remain in solution and be delivered into the amnion of poultry eggs by in ovo injection. The carbohydrate may be a glucan or a combination of various glucans as shown in the Examples. Branched or linear glucans may be used. Suitable glucans include dextrin, dextran and starches. Suitable dextrins include maltodextrin and suitable starches include potato starch.

Without being limited by theory, the carbohydrate is likely providing an additional source of nutrients for the embryo/hatchling. The carbohydrates used may also be a sink for any free unbound iodine or iodine containing molecules. The amount of the carbohydrate in the composition is varied to provide a composition that is approximately isotonic with the egg. Suitably the carbohydrate is added to the composition such that it is 15-40% of the composition, suitably more than 17%20%, 22%, 24%, 25%, 27%, 28%, or 29%. Suitably the carbohydrate is less than 40%, 37%, 35%, 33%, 31%, or 30%. The percentages may be combined in any combination to derive suitable ranges for the composition. In the Examples, a total carbohydrate concentration is a solution of 28% carbohydrate consisting of 1.8% maltodextrin and 10% potato starch.

The composition is a solution that is approximately isotonic or hypotonic as compared to the poultry embryo in ovo. The osmolality of a poultry egg is about 300 mOsm. The composition suitably has an osmolality greater than 100 mOsm, or more suitably greater than about 150 mOsm, 200 mOsm, 250 mOsm, 275 mOsm, 285 mOsm, 295 mOsm or 300 mOsm. Suitably the composition has an osmolality of less than about 450 mOsm, or more suitably less than 400 mOsm, 350 mOsm; 325 mOsm, 315 mOsm, 310 mOsm, 305 mOsm, or 300 mOsm. The osmolarities may be combined in any combination to derive suitable ranges for the composition.

The composition may also include or be used as a earner for antibiotics, vaccines, proteins, saline solutions, buffering solutions, nutraceuticals, pharmaceuticals or other additives. The composition may be used as a carrier for other pharmaceutical or nutraceutical compositions being administered in ovo. Compositions such as those described above may include a pharmaceutically acceptable carrier. A pharmaceutically acceptable carrier is any carrier suitable for in vivo or in ovo administration. Examples of pharmaceutically acceptable carriers suitable for use in the composition include, but are not limited to, water, buffered solutions, glucose solutions, oil-based or bacterial culture fluids. Additional components of the compositions may suitably include, for example, excipients such as stabilizers, preservatives, diluents, emulsifiers and lubricants. Examples of pharmaceutically acceptable carriers or diluents include stabilizers such as carbohydrates (e.g. sorbitol, mannitol, starch, sucrose, glucose, or dextran), proteins such as albumin or casein, protein-containing agents such as bovine serum or skimmed milk and buffers (e.g., phosphate buffer). Especially when such stabilizers are added to the compositions, the composition is suitable for freeze-drying or spray-drying for later reconstitution.

The composition may be administered to poultry embryos in ovo to increase the health of poultry hatchlings. The poultry hatchlings include but are not limited to chicken and turkey hatchlings, but are expected to improve the hatch health and hatchability of any egg, including duck, goose and other fowl. The composition may be injected into the eggs by hand or by mechanized injection systems used in the poultry industry. Such methods are used for vaccination and are well known to those of skill in the art.

The composition may be administered between 1-3 days prior to hatch. Suitably the composition is administered 2 days prior to hatch. For turkey poults, administration around day 25 of embryonic development is suitable for administration of the composition. For layer or broiler eggs about day 18 of incubation is suitable for administration of the composition. Eggs are often transferred from the incubation units to the hatching unit about this time and thus the timing is convenient for commercial breeding operations.

The dose of the composition administered to the embryos will vary depending on the constituents of the composition. The amount of iodinated tyrosine-containing protein in the composition administered per gram of poultry embryo is between 0.1 μg/g and 5 μg/g. Suitably at least 0.2 μg/g, 0.3 μg/g, 0.4 μg/g or 0.5 μg/g of the iodinated tyrosine-containing protein is administered to each embryo. Suitably no more than 4.5 μg/g, 4.0 μg/g, 3.5 μg/g, or 3.0 μg/g of the iodinated tyrosine-containing protein is administered to each embryo. Based on the average weights of turkey and chicken eggs, the amount of iodinated tyrosine-containing protein administered ranges between 1 and 200 μg/egg. Suitably at least 2, 5, 7, 10, 15 or 20 μg/egg and not more than 175, 150, 125,100, or 75 μg/egg of the iodinated tyrosine-containing protein is administered. Suitably the total volume administered per egg is less than 500 μL, 400 μL, 350 μL, 300 μL, 250 μL, or 200 μL. The volume administered in the examples was 200 μL. Suitably the volume administered is more than about 5 μL, 10 μL, 20 μL, 50 μL, 75 μL, 100 μL.

The administration of the composition may increase the health of poultry hatchlings in a number of measurable ways. Administration increases the hatchability of poultry. The hatchability refers to the number or percentage of the eggs giving rise to viable hatchlings. The percent increase in hatchlings may be 1%, 2%, 3%, 4% or more as compared to control animals injected with diluent alone.

Administration of the composition may also result in an increase in hatchling weight at hatch. The increase in hatchling weight at hatch may be greater in hatchlings in the middle 50% or the bottom 25% of weights as compared to their cohort. The increase may be a 0.5 g, 1 g, 2 g, 3 g or greater increase in body weight at hatch as compared to control animals injected with diluent alone.

Administration of the composition may also result in an increase in hatchling weight at 7 days post-hatch. The increase in hatchling weight at 7 days post-hatch may be greater in hatchlings in the middle 50% or the bottom 25% of hatch weights as compared to their cohort. The increase may result in a 0.5 g, 1 g, 2 g, 3 g or greater increase in body weight at 7 days post-hatch as compared to control animals injected with diluent alone.

Administration of the composition may also result in a decrease in mortality post-hatch. The decrease in mortality post-hatch may be greater in hatchlings in the middle 50% or the bottom 25% of weights as compared to their cohort. The decrease may be a 0.5%, 1%, 2%, 3% or more decrease in mortality post-hatch as compared to control animals injected with diluent alone.

### EXAMPLES

### Example 1

### Turkey Poults

### Materials and Methods

### Iodinated Casein Solution

An iodinated casein solution was synthesized according to Kuenzel et al. (2005, World's Poult. Sci. 61.599-624). Briefly, 1 L of distilled water containing 238 mg of MnSO₄¹ (2.8 mmoles/mL) and 22.5 g NaHCO₃ (154 mmoles/mL) was heated to 40° C. and 6.75 g casein was slowly added to this solution with gentle stirring. Following this step, 12.4 g potassium iodide (8.7 mmoles) was added and heated in a water bath at 70° C. overnight (˜20 to 24 h). A portion of this iodinated casein mixture was added to one liter of a solution containing 10 mg/mL gentamycin and a carbohydrate solution composed of 18% maltodextrin and 10% potato starch dextrin (280-290 mOsm). This dextrin mixture was derived from an initial pilot study (see below) as well as testing the osmolarity of several other dextrin solutions singly or in various combinations. In the two studies described below, the final concentrations of iodinated casein were calculated to contain 75 μg/mL (1×) or 375 μg/mL (5×) iodinated casein (IC) per mL of solution (IC75 and IC375, respectively). Controls received a physiological saline injection containing gentamycin (10 mg/mL) which was being used at the time in the commercial hatchery to test in ovo vaccination of turkey embryos. ¹All chemicals were obtained from Sigma Chemical Co., St. Louis Mo.

### Egg Injections

The experiments were conducted at a local commercial turkey hatchery that produces approximately 280,000 poults per week. The injections were carried out on turkey eggs on day 25 of incubation using a commercial egg injector system (in Ovoject, Embrex, Raleigh, N.C.) capable of injecting 50 turkey eggs at a time. Control and iodinated casein treatments were injected into eggs at 200 μL per egg. The injections and transfer of eggs to commercial hatcher units were performed by hatchery personnel.

### Hatchability and Poult Weights

Hatchability in both Exp. 1 and 2 was determined by hatchery personnel at 72 h post-injection (at 28 days of incubation) in hatching trays containing 100 eggs per tray. In Exp. 1, the eggs were obtained from a mature breeder flock that was 33 weeks into production whereas eggs in Exp. 2 were obtained from a young breeder flock that was 5 weeks into production.

In Exp. 1, a total of 3900 eggs were injected with the Control solution, or with the Dextrin (Dex) solution with 75 or 375 μg/mL of iodinated casein (DexIC75 and DexIC375, respectively) solution (1300 eggs per group). The eggs were placed in trays of 100 eggs per tray, the trays marked for identification, and placed in a single hatcher unit. A total of 600 poults (200 poults per group) were neck-tagged for identification, hatch weights (g) obtained, transported to a commercial contract grower and placed in a single brooder ring in a commercial turkey house. At 7 days post-hatch, the poults were weighed, their neck tags removed, and then released with other poults when brooder rings were taken up in the house.

In Exp. 2, 5200 eggs received either the Control (2600 eggs) or the DexIC75 solution (2600 eggs). The eggs were placed in egg trays, the trays marked for identification and placed in 2 different hatcher units (1300 eggs per group per hatcher unit). Hatchability was determined 72 hours later, and 600 poults (300 per group) were randomly selected, neck-tagged, weighed, transported to a grower and placed with a single brooder ring. The poults were weighed 6 days later and neck tags removed when the brooder rings were taken up in the commercial turkey house.

### Statistical Analyses

Data for each group are presented as the mean±SE, and the means were compared by t-test using JMP² statistical analysis software. Data are shown for the entire treatment group as well as the top and bottom 25% and middle 50% with the treatment population. As poults were held in the same pen, individual weights were used as the statistical unit. A probability level of P≦0.05 was considered statistically significant.

### Results

A pilot was conducted to test the effects of several dextrin solutions alone and in combination with iodinated casein on turkey hatchability to determine the best dextrin and iodinated casein levels, and to insure that the solutions did not adversely affect turkey hatchability. Based on these results, the best carbohydrate solution appeared to be 18% maltodextrin and 10% potato starch dextrin (Dex) with 75 and 375 μg/mL iodinated casein (IC75 and IC375). In this study, hatchability was improved 3-5% after injection with dextrin in combination with iodinated casein as compared to controls.

In Exp. 1, there was no difference in hatchability between the Control, DexIC75 and DexIC375 treatment groups although hatchability was numerically higher in the DexIC75 group (Table 1). The DexIC75 treated poults were heavier (P=0.028) at hatch with a 1.5 g difference observed in birds that were in the top 25% compared to the Control group (Table 1). There were no differences in 7 day body weights, 7 day gain or average daily gain between treatment groups, however.

TABLE 1
The effects of in ovo injections (200 μL) into turkey eggs on
day 25 of incubation of a Control solution or solutions containing
dextrin (Dex; 18% maltodextrin and 10% potato starch dextrin) in
combination with iodinated casein (IC) at either 75 μg/mL (IC75)
or 375 μg/mL (IC375) on hatchability, hatch weight, 7 day body
weight (BW), 7 day gain and average daily gain (ADG).

| | Hatchability | |
|---|---|---|
| | (%) | (n¹) |
| Control | 91.9 ± 1.0 | 13 |
| DexIC75 | 92.5 ± 0.6 | 13 |
| DexIC375 | 91.7 ± 1.0 | 13 |

| Treatment | Top 25% | Middle 50% | Bottom 25% | Overall |
|---|---|---|---|---|
| Control (n) | (50) | (97) | (47) | (194) |
| DexIC75 (n) | (50) | (97) | (42) | (189) |
| DexIC375 | (50) | (97) | (45) | (192) |
| (n) | | | | |
| | Hatch Weight (g) | | | |
| Control | 56.6 ± 0.6 | 54.9 ± 0.4 | 53.5 ± 0.5 | 55.0 ± 0.3b |
| DexIC75 | 58.0 ± 0.6 | 55.6 ± 0.4 | 54.1 ± 0.6 | 55.9 ± 0.3a |
| DexIC375 | 57.2 ± 0.6 | 55.5 ± 0.4 | 54.0 ± 0.6 | 55.7 ± 0.3ab |
| Con vs. | | | | (P = 0.028) |
| DexIC75 | | | | |
| Con vs. | | | | (P = 0.093) |
| DexIC375 | | | | |
| | 7 d BW (g) | | | |
| Control | 166.1 ± 1.2 | 147.8 ± 0.5 | 127.4 ± 1.9 | 147.6 ± 1.0 |
| DexIC75 | 166.6 ± 0.9 | 147.7 ± 0.6 | 127.4 ± 1.1 | 148.2 ± 1.1 |
| DexIC375 | 167.0 ± 1.1 | 148.8 ± 0.6 | 125.2 ± 1.5 | 148.0 ± 1.2 |
| | 7 d Gain (g) | | | |
| Control | 109.5 ± 1.2 | 92.9 ± 0.6 | 74.0 ± 2.0 | 92.6 ± 1.0 |
| DexIC75 | 108.6 ± 0.9 | 92.2 ± 0.7 | 73.0 ± 1.2 | 92.4 ± 1.0 |
| DexIC375 | 109.9 ± 1.2 | 93.2 ± 0.7 | 71.3 ± 1.6 | 92.6 ± 1.1 |
| | ADG (g/d) | | | |
| Control | 15.6 ± 0.2 | 13.3 ± 0.1 | 10.6 ± 0.3 | 13.2 ± 0.2 |
| DexIC75 | 15.5 ± 0.1 | 13.2 ± 0.1 | 10.4 ± 0.2 | 13.2 ± 0.2 |
| DexIC375 | 15.7 ± 0.2 | 13.3 ± 0.1 | 10.2 ± 0.2 | 13.2 ± 0.2 |

¹Hatchability determined as % on 100 eggs per tray.

In Exp. 2, hatchability was 2.6% higher (P=0.011) and poults were heavier at hatch (P<0.0001) in the IC75 group compared to Controls (Table 2). The heavier hatch weights of poults in the DexIC75 group was due to increases in hatch weight in both the top 25% (P=0.0021) and middle 50% (P=0.0044) of the poults (Table 2). Unlike Experiment 1, DexIC75 treated poults were heavier (P=0.03) on day 6 compared to the Control group. There were no differences in average daily gain between groups, however.

TABLE 2
The effects of in ovo injections into turkey eggs after 25 days of
incubation of a Control solution or a solution containing 18%
maltodextrin and 10% potato starch dextrin (Dex) in combination
with 75 μg/mL iodinated casein (IC) on hatchability, hatch weight,
6 day body weight (BW), 6 day gain and average daily gain (ADG).

| | Hatchability | |
|---|---|---|
| Treatment | (%) | (n¹) |
| Control | 84.5 ± 0.8 | 26 |
| DexIC75 | 86.9 ± 0.6 | 26 |
| P Value | = 0.011 | |

| Treatment | Top 25% | Middle 50% | Bottom 25% | Overall |
|---|---|---|---|---|
| Control (n) | (73-75) | (143-150) | (72-75) | (186-200) |
| DexIC75 (n) | (73-75) | (147-150) | (73-75) | (191-200) |
| | Hatch Weight (g) | | | |
| Control | 57.6 ± 0.5 | 56.5 ± 0.5 | 56.1 ± 0.5 | 56.7 ± 0.3 |
| DexIC75 | 60.0 ± 0.5* | 57.9 ± 0.3* | 56.9 ± 0.5 | 58.2 ± 0.3* |
| P Value | = 0.0021 | = 0.0044 | >0.10 | <0.0001 |
| | 6 d Body Weight (g) | | | |
| Control | 125.8 ± 0.6 | 110.6 ± 0.4 | 95.1 ± 0.8 | 110.8 ± 0.7 |
| DexIC75 | 128.2 ± 0.6* | 113.3 ± 0.4 | 97.0 ± 0.9 | 113.0 ± 0.7* |
| P Value | = 0.016 | <0.0001 | >0.10 | = 0.03 |
| | 6 d Gain (g) | | | |
| Control | 68.4 ± 0.8 | 54.1 ± 0.5 | 39.4 ± 0.9 | 54.2 ± 0.7 |
| DexIC75 | 68.1 ± 0.7 | 55.4 ± 0.5* | 40.2 ± 1.0 | 54.9 ± 0.7 |
| P Value | >0.10 | = 0.061 | >0.10 | >0.10 |
| | ADG (g/d) | | | |
| Control | 11.4 ± 0.1 | 8.9 ± 0.1 | 6.6 ± 0.1 | 9.0 ± 0.1 |
| IC75 | 11.4 ± 0.1 | 9.2 ± 0.1* | ‘6.7 ± 1.0 | 9.1 ± 0.1 |
| P Value | >0.10 | = 0.068 | P > 0.10 | >0.10 |

¹Hatchability determined on 100 eggs per tray
*Indicates significantly different mean values at the probability shown.

In the present study, under commercial turkey production practices, in ovo feeding of a dextrin solution containing 18% maltodextrin and 10% potato starch dextrin in combination with 75 μg/ml, iodinated casein (DexIC75) improved hatchability, poult hatch weight, and/or subsequent early growth. To our knowledge, this is the first time that such treatments have been tested in turkey embryos.

Detailed studies that could include biochemical and molecular analysis will be required in order to specifically characterize responses of embryos to dextrin and iodinated casein independently, Regardless of the mechanisms involved, the results of the present study demonstrate that a solution containing 18% maltodextrin and 10% potato starch dextrin in combination with 75 μg/ml, of iodinated casein appears to be beneficial to hatchability and early post-hatch growth under commercial turkey hatchery and production conditions.

### Example 2

### Broiler Chicks

Similar experiments to those described in Example 1 were completed in broilers to assess whether the improvement in hatchability and early weight gain could be replicated in chickens. A total of 1200 eggs were injected with the Control solution, or with the Dextrin (Dex) solution alone or with the Dex solution with 75 or 375 μg/mL of iodinated casein (DexIC75 and DexIC375, respectively) solution or with 75 μg/mL of iodinated casein alone (200 eggs per group). The eggs were placed in trays of 100 eggs per tray, the trays marked for identification, and placed in a single hatcher unit. A total of 150 chicks per group were neck-tagged for identification, hatch weights (g) obtained, transported to a commercial contract grower and placed in a single brooder ring in a commercial broiler house. At 7 days, the chicks were weighed, their neck tags removed, and then released with other chicks when brooder rings were taken up in the house.

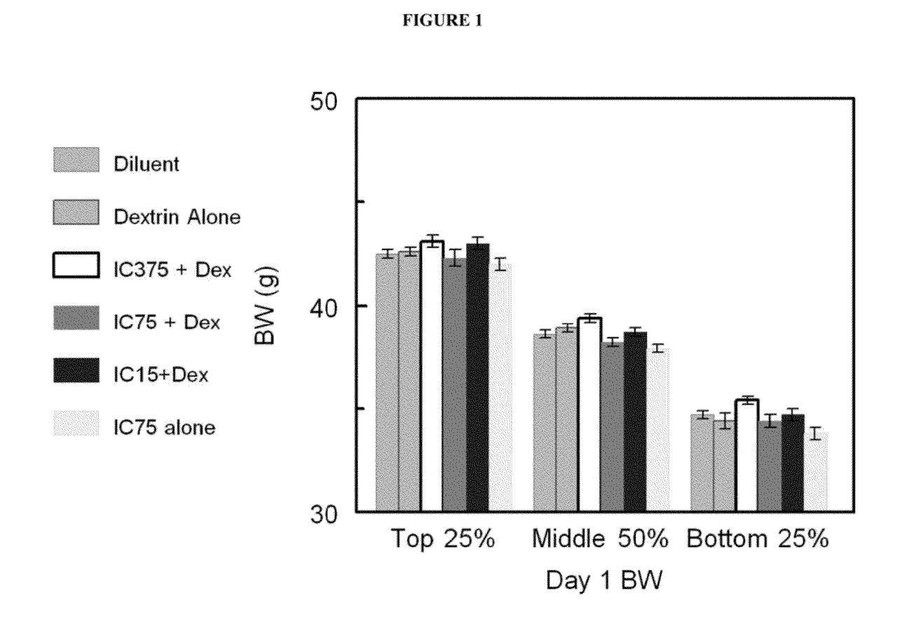

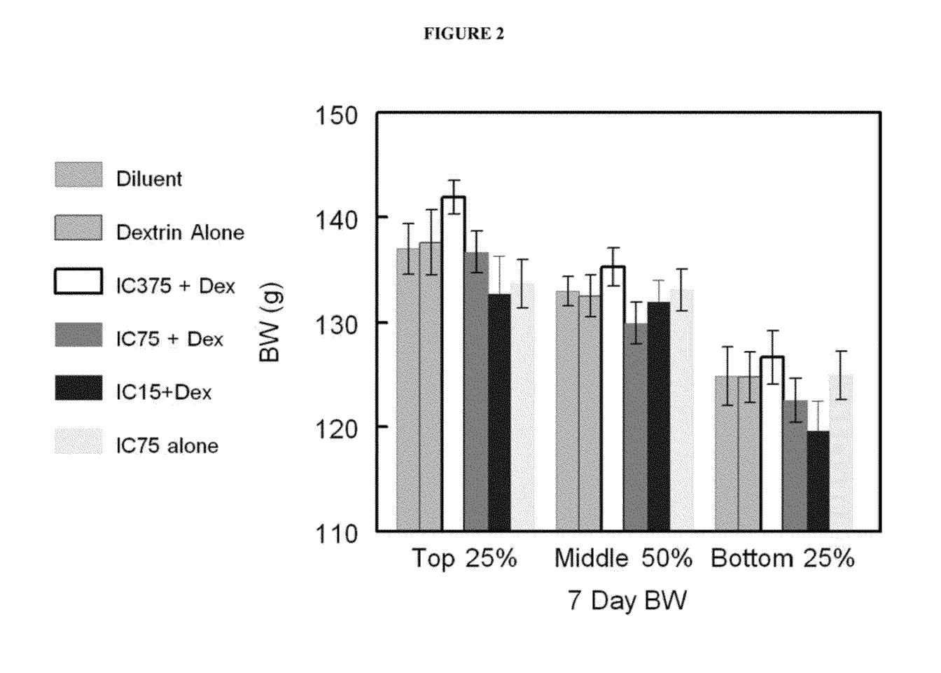

In this study, broiler chicks in either the middle 50% or the bottom 25% of hatch weight were heavier at hatch when administered DexIC375 than controls given diluent only (P=0.001 for middle 50%; P=0.09 for bottom 25%). See FIG. 1. At day 7 post-hatch, the chicks receiving DexIC375 and in the top 25% of the group were somewhat heavier than those receiving diluent alone (P=0.092). See FIG. 2. As with turkeys, in ovo feeding of a dextrin solution containing 18% maltodextrin and 10% potato starch dextrin in combination with iodinated casein improved hatch weight, and possibly subsequent early growth.

1. A composition comprising: an iodinated tyrosine-containing protein and a carbohydrate.

2. The composition of claim 1, wherein the composition has an osmolality between 150 mOsm and 450 mOsm.

3. The composition of claim 1, wherein the composition has an osmolality between 200 mOsm and 400 mOsm.

4. The composition of claim 1, wherein the composition has an osmolarity between 250 mOsm and 350 mOsm.

5. The composition of claim 1, wherein the carbohydrate is soluble at 4° C.

6. The composition of claim 1, wherein the composition is injectable.

7. The composition of claim 1, wherein the carbohydrate is selected from glucans, dextrin, starch or combinations thereof.

8. The composition of claim 7, wherein the starch is potato starch.

9. The composition of claim 7, wherein the dextrin is maltodextrin.

10. The composition of claim 1, wherein the iodinated tyrosine-containing protein is casein.

11. The composition of claim 1, wherein the composition comprises between 10 μg/ml and 1000 μg/ml of the iodinated tyrosine-containing protein.

12. The composition of claim 1, wherein the composition comprises between 50 μg/ml and 4800 μg/ml of the iodinated tyrosine-containing protein.

13. A method of increasing the health of poultry hatchlings comprising administering a composition comprising an iodinated tyrosine-containing protein to poultry eggs in ovo, wherein the health of the hatchling is increased as compared to a control and wherein the iodinated tyrosine-containing protein is casein.

14. A method of increasing the health of poultry hatchlings comprising administering the composition of claim 1 to poultry embryos in ovo.

15. The method of claim 14, wherein the composition is administered at about 1-3 days prior to hatch.

16. The method of claim 14, wherein the composition is administered about 2 days prior to hatch.

17. The method of claim 14, wherein the poultry embryos are turkey or chicken embryos.

18. The method of claim 14, wherein the increase in health of the hatchlings comprises at least one of an increase in the percent of eggs that hatch as compared to a control, an increase in hatchling weight at hatch as compared to a control, an increase in weight gain over the first week post-hatch as compared to a control, or a decrease in mortality post-hatch as compared to a control.

19. (canceled)

20. (canceled)

21. (canceled)

22. The method of claim 14, wherein the amount of iodinated tyrosine-containing protein in the composition administered per gram of poultry embryo ranges from about 0.03 μg to about 3.0 μg/g embryo.

23. The method of claim 14, wherein the amount of iodinated tyrosine-containing protein in the composition administered ranges from about 2 to about 100 μg/egg.
